(19) United States
(12) Patent Application Publication
(10) Pub. No.: US 2013/0302255 A1
(43) Pub. Date: Nov. 14, 2013

(54) NOVEL TARGETED PARAMAGNETIC CONTRAST AGENT

(71) Applicant: BBS NANOMEDICINA ZRT., Debrecen (HU)

(72) Inventors: János BORBÉLY, Debrecen (HU); István HAJDÚ, Tiszacsege (HU); Magdolna BODNÁR, Debrecen (HU); Ildikó SCRIFFERTNÉ DENYICSKA, Debrecen (HU)

(21) Appl. No.: 13/888,675

(22) Filed: May 7, 2013

Related U.S. Application Data
(60) Provisional application No. 61/644,505, filed on May 9, 2012.

(51) Int. Cl. A61K 49/08; A61K 49/18; A61K 49/12

(52) U.S. Cl. CPC A61K 49/085; A61K 49/12; A61K 49/1818
USPC 424/9.323; 424/9.3; 424/9.35

(57) ABSTRACT

Disclosed are novel, targeting, paramagnetic nanoparticles as contrast agent for magnetic resonance imaging. The compositions of the nanoparticles are composed of self-assembled polyelectrolyte biopolymers having targeting moieties, which can suitable for targeted delivery of paramagnetic ions complexed to the nanoparticles. The nanoparticulate contrast agent can internalize into the targeted tumor cells to realize the receptor mediated uptake, and therefore afford enhanced relaxivity and improved signal-to-noise effect on the examined tissue areas. Methods for making these targeting MRI contrast agents are also provided.

### BRIEF DESCRIPTION OF THE INVENTION

The present invention discloses novel, targeting, paramagnetic nanoparticles as contrast agent for magnetic resonance imaging. The compositions of the nanoparticles are composed of self-assembled polyelectrolyte biopolymers having targeting moieties, which can be suitable for the targeted delivery of paramagnetic ions complexed to the nanoparticles. The nanoparticulate contrast agent can internalize into the targeted tumor cells to realize the receptor mediated uptake, and therefore afford enhanced relaxivity and improved signal-to-noise effect on the examined tissue areas. Methods for making these targeting MRI contrast agents are also provided.

### REFERENCES CITED

### Patent Documents

| | | |
|---|---|---|
| U.S. Pat. No. 6,562,802 B2 | May 2003 | Johansson et al. |
| U.S. Pat. No. 6,896,874 B2 | May 2005 | Li et al. |
| U.S. Pat. No. 8,007,768 B1 | August 2011 | Sung et al. |
| U.S. Pat. No. 8,048,404 B1 | November 2011 | Sung et al. |
| U.S. Pat. No. 8,048,453 B1 | November 2011 | Sung et al. |
| U.S. Pat. No. 8,084,493 B1 | December 2011 | Sung et al. |
| US 2007/0196275 A1 | August 2007 | Li et al. |
| US 2006/0251580 A1 | November 2006 | Keppler et al. |
| US 2010/0069293 A1 | March 2010 | Bolotin et al. |
| US 2007/0129792 A1 | June 2007 | Picart et al. |
| US 2007/0122342 A1 | May 2007 | Yang et al. |
| US 2007/0292387 A1 | December 2007 | Jon et al. |
| US 2002/0197261 A1 | December 2002 | Li et al. |
| US 2008/0171070 A1 | July 2008 | Schaaf et al. |
| U.S. Pat. No. 7,976,825 B2 | July 2011 | Borbely et al. |
| U.S. Pat. No. 7,291,598 B2 | November 2007 | Sung et al. |
| WO/1998/026788 | June 1998 | Johansson et al. |
| WO 2004/096998 | November 2004 | Prokop et al. |

### OTHER PUBLICATIONS

Vincent Darras, Monica Nelea, Francoise M. Winnik, Michael D. Buschmann, Chitosan modified with gadolinium diethylenetriaminepentaacetic acid for magnetic resonance imaging of DNA/chitosan nanoparticles, Carbohydrate Polymers 80 (2010) 1137-1146.
Prashant Agrawal, Gustav J. Strijkers, Klaas Nicolay, Chitosan-based systems for molecular imaging, Advanced Drug Delivery Reviews 62 (2010) 42-58.
Min Huang, Zhixin L. Huang, Mehmet Bilgen, Cory Berkland, Magnetic resonance imaging of contrast-enhanced polyelectrolyte complexes, Nanomedicine: Nanotechnology, Biology, and Medicine 4 (2008) 30-40.
Ching Ting Tsao, Chih Hao Chang, Yu Yung Lin, Ming Fung Wu, Jaw-Lin Wang, Jin Lin Han, Kuo Huang Hsieh, Antibacterial activity and biocompatibility of a chitosan-γ-poly(glutamic acid) polyelectrolyte complex hydrogel, Carbohydrate Research 345 (2010) 1774-1780.
Kiran Sonaje, Yi-Jia Chen, Hsin-Lung Chen, Shiaw-Pyng Wey, Jyuhn-Huarng Juang, Ho-Ngoc Nguyen, Chia-Wei Hsu, Kun-Ju Lin, Hsing-Wen Sung, Enteric-coated capsules filled with freeze-dried chitosan/poly(γ-glutamic acid) nanoparticles for oral insulin delivery, Biomaterials 31 (2010) 3384-3394.
Ching Ting Tsao, Chih Hao Chang, Yu Yung Lin, Ming Fung Wu, Jaw Lin Wang, Tai Horng Young, Jin Lin Hane, Kuo Haung Hsieh, Evaluation of chitosan/γ-poly(glutamic acid)polyelectrolyte complex for wound dressing materials Carbohydrate Polymers 84 (2011) 812-819.
Hua Ai, Layer-by-layer capsules for magnetic resonance imaging and drug delivery, Advanced Drug Delivery Reviews 63 (2011) 772-788.
Guo-Ping Yan, Leslie Robinson, Peter Hogg, Magnetic resonance imaging contrast agents: Overview and perspectives, Radiography 13 (2007) e5-e19.
Chien-YangHsieh, Sung-Pei Tsai, Da-MingWang, Yaw-Nan Chang, Hsyue-Jen Hsieh, Preparation of γ-PGA/chitosan composite tissue engineering matrices, Biomaterials 26 (2005) 5617-5623.
Yu-Hsin Lin, Ching-Kuang Chung, Chiung-Tong Chen, Hsiang-Fa Liang, Sung-Ching Chen, Hsing-Wen Sung, Preparation of nanoparticles composed of chitosan/poly-γ-glutamic acid and evaluation of their permeability through Caco-2 cells, Biomacromolecules 6 (2005) 1104-1112.

This application takes the priority of U.S. Provisional Patent Application Ser. No. 61/644,505, filed on the 9^{th} of May, 2012, the entire content of which is incorporated herein by reference.

### FIELD OF THE INVENTION

The present invention relates to self-assembled nanoparticles having a composition of chitosan polycation or its derivatives meaning the covalent conjugate of complexing agent thereto, polyanion, and targeting agent covalently conjugated to one of the polyelectrolete biopolymers, and paramagnetic ions for diagnostic applications on the field of magnetic resonance imaging (MRI). The novel targeting nanoparticles as MRI contrast agents and methods for their production and use are also related.

### BACKGROUND OF THE INVENTION

Magnetic resonance imaging (MRI) is an imaging technique used primarily in medical settings to produce high quality images of the inside of the human body. MRI is based on the principles of nuclear magnetic resonance (NMR), a spectroscopic technique used by scientists to obtain microscopic chemical and physical information about molecules. MRI started out as a tomographic imaging technique, that is it produced an image of the NMR signal in a thin slice through the human body. MRI has advanced beyond a tomographic imaging technique to a volume imaging technique.

Magnetic resonance imaging is based on the absorption and emission of energy in the radio frequency range of the electromagnetic spectrum. It uses no ionizing radiation, but uses a powerful magnetic field to align the nuclear magnetization of some atomic nuclei, for example hydrogen atoms in water in the body. The human body consists of primarily fat and water. Fat and water have many hydrogen atoms which make the human body containing approximately 63% by weight hydrogen atoms. Radiofrequency fields are used to systematically alter the alignment of the nuclear magnetization of hydrogen atoms. The hydrogen nuclei are caused to produce a rotating magnetic field detectable by the scanner- and this information is recorded to construct an image of the scanned area of the body. The body is mainly composed of water molecules which each contain two hydrogen nuclei or protons. When a person goes inside the powerful magnetic field of the scanner these protons align with the direction of the field. A second radiofrequency electromagnetic field is then briefly turned on causing the protons to absorb some of its energy. When this field is turned off the protons release this energy at a radiofrequency which can be detected by the scanner. The position of protons in the body can be determined by applying additional magnetic fields during the scan which allows an image of the body to be built up.

Magnetic resonance imaging (MRI) is one of the most important diagnostic imaging techniques. Advantages of MRI are that it is a non-invasive, versatile diagnostic methodology in clinical radiology, and it provides excellent soft-tissue imaging contrast. MRI is primarily used for visualization of anatomic details of soft tissues and organs. The signal of MRI depends on the longitudinal (T1) and transversal (T2) relaxation times of water and therefore changes of relaxation times results in difference of signal, which appears in contrast in MR images. MRI has developed rapidly and has become especially useful method in the diagnosis and medication of neurological, cardiovascular and oncological diseases.

It is important that images are hard-contrast and sensitive with high stereoscopic resolution. Pathological changes can be detected via physico-chemical differences, which can be exploited through varying light intensities (on the grey scale). The image contrast means a detectable difference between signal intensities, which induces optical stimulus thereby making diagnosis possible.

Nowadays, much attention is given to use and develop intravascular contrast agents to enhance the sensitivity and resolution of MRI and to provide excellent soft-tissue contrast. Superparamagnetic and paramagnetic materials can be used as contrast agent. They can change the homogeneity of the magnetic field and alter the relaxation time of the tissue water, where they reside, and producing significant contrast difference between the examined and surrounding tissues. Contrast agents of these characters could be iron oxide nanoparticles, gadolinium chelates or manganese-based materials

Paramagnetic contrast agents are designed to reduce the T1 (longitudinal) relaxation time of water and therefore result in the difference of signal, and in consequence in contrast in MR images. The targeted tissue signal is strengthened and looks brighter in the MR images.

Many recent attempts have been made to create paramagnetic compositions for application as sensitive contrast agents for MRI. In the most cases these compositions usually contain Gd-ions, Mn-ions, or their complexes.

Recently, both small molecular and macromolecular contrast agents have attracted much interest because of their ability to improve MRI signals. Small molecular contrast agents have been used successfully for contrast enhancement of MR imaging. However, these compositions are non-specific extracellular contrast agents and have serious shortcomings, such as short half-life time in blood, rapidly diffuse out of the blood and excrete thought the kidney resulting in low image quality, lack of targeting specificity and limited use in other parts of the body.

To eliminate these shortcomings, several nanoparticular contrast agents have been developed. Liposomes, microbubbles, metallofullerens, carbon nanotubes and several dendritic nanodevices were engineered to serve as contrast agents for MRI. Numerous recent attempts have been made to create macromolecule-based sensitive carriers for contrast enhancement of MRI. Polymeric micelles, polyelectrolyte complexes, proteins, polysaccharides, water-soluble fullerenes, and other biocompatible natural and synthetic polymer have been investigated as potential carriers of contrast agents for MR imaging modality.

The macromolecular contrast agents have several advantages. Due to their colloid size, they circulate in the blood for a long time; therefore significant contrast can be obtained over a long period of time. In addition, these systems can be modified flexibly via their functional groups, and multiple, targeted nanocarriers can be formed.

Hydrophilic polymers, macromolecules can behave as polyelectrolyte due their charged functional groups in aqueous media. Based on the attractive interaction of oppositely charged functional groups of polyelectrolytes can self-assembly and can result in stable polyelectrolyte complexes. The polyelectrolyte complexes dispose several advantages, such as numerous reactive functional groups, the flexibility of the system and a lack of new covalent bond, which could modify the favorable biological properties of biopolymers.

Self-assembly of polyelectrolytes produces stable polyelectrolyte complexes, which can appear in nanoparticles, nanosystems, films or hydrogels. A variety of studies have focused on preparation and characterization of these polyelectrolyte complexes, because these systems open many new opportunities to develop delivery of bioactive molecules. Several polyelectrolyte complex systems were developed for use as carrier for drug or gene delivery.

After self-assembly, the residual functional groups of polyelectrolytes are available for transport, and for targeting of active agents.

In clinical use, the contrast agents suffer from disadvantages, e.g. non-specificity or evoking of side effects. In order to achieve optimal contrast, a certain dose is required. On the other hand, the dose applicable to the body is limited, because allergic reactions in the recipient must be avoided. Therefore, there is an increasing interest and need for development of novel, specific, targeted MRI contrast agents.

Targeting MRI contrast agents internalize and accumulate selectively in the targeted specific cells, tissues, therefore a smaller dose is sufficient to increase the signal difference between the examined and surrounded tissue areas. These systems contain active targeting molecule, which enable the specific binding and receptor mediated uptake of contrast agent into the targeted tumor cells.

Ideally, a polymer-based MRI contrast agent and accumulates in the targeted tumor cell. Small dose of targeted contrast agent to produce visible hard-contrast in MRI and to allow completion of the imaging procedure; afterwards it should be degraded and excreted through the kidneys.

Ideally, contrast agents are specific, reside in the blood, circulate in the body for a long time, target and localize to the tumor cell, achieved hard-contrast between the examined tumor and healthy sites, and to allow of prolonged duration of MR imaging. Due to the significant accumulation of tumor-specific MRI contrast agents in the targeted tumor cell, lower doses are sufficient to increase the signal difference between the targeted tissues and the background. These systems contain active targeting moiety, which enables the specific binding and internalization of contrast agent into the targeted tumor cells.

Targeting ligands include small molecules (e.g. folic acid), peptides (e.g. LHRH), monoclonal antibodies (e.g. Transtuzumab) or others.

Folic acid is a widely used targeting moiety of carrier for cancer therapy. It has been shown, that several human tumor cells overexpress folate receptors, and possess a high affinity for folic acid molecules. However normal tissues possess restricted number of folate receptors.

Chitosan (CH) is a renewable, basic linear polysaccharide, containing β[1→4]-linked 2-acetamido-2-deoxy-D-glucopyranose and 2-amino-2-deoxy-D-glucopyranose units with reactive amino groups. Because of its special set of properties, which include low or non-toxicity, biocompatibility, biodegradability, low or no immunogenicity and antibacterial properties, chitosan has found wide application in a variety of areas, such as biomedicine, pharmaceuticals, metal chelation, food additives, and other industrial applications. Its application could be difficult because of its low solubility in aqueous media. Chitosan can be solubilized by the protonation of its amino groups in acidic media, resulting in a cationic polysaccharide with high charge density appearing in viscous solution.

Poly-γ-glutamic acid (γ-PGA) consists of repetitive glutamic acid units connected by amide linkages between α-amino and γ-carboxylic acid functional groups. Γ-PGA is different from other proteins, in that glutamate is polymerized via the non-peptide γ-amide linkages, and thus is synthesized in a ribosome-independent manner. In could be prepared by bacterial fermentation with molecular weight range between 10 kDa and 1000 kDa.

Γ-PGA is a hydrophilic, water soluble, biodegradable, edible and nontoxic polypeptide. It is a polyanion having reactive carboxyl groups; it is non-toxic for the environment and humans. Therefore, γ-PGA and its derivatives have been employed extensively in a variety of commercial applications such as cosmetics, food, medicine, and water treatment.

### BACKGROUND ART

Johansson et al. (U.S. Pat. No. 6,562,802) describe a composition based on the native chitosan, which is not covalently bound to DTPA, DOTA, or EDTA, or other chelators. The composition is used as a medicament in a topical barrier formulation, an UV radiation absorbing formulation, and an antiviral, antifungal or anti-inflammatory formulation. The composition can make stable complex with allergens and/or irritating agents, such as Ni²⁺, Ci³⁺, Cr⁶⁺, Co²⁺, Au⁺, and Au³⁺ ions. This work deals only with chitosan biopolymer and the linkage between the biopolymer and the complexone is not covalent. The inventors do not prepare particles, do not work with Gd-ions and do not target.

Li et al. (U.S. Pat. No. 6,896,874 B2) describe a coating that emits magnetic resonance signals. The coating includes paramagnetic metal ion containing polymer complex. The invention claimed paramagnetic-metal ion/chelate complexes encapsulated by a hydrogel. The chelate could be several well-known complexing agents, such as DTPA or DOTA, and the paramagnetic ion could be lanthanide or transition metal ions, such as Gd-ion. The first hydrogel is selected from chitosan hyaluronate, alginate, poly(acrylic acid), etc.

Sung et al. (U.S. Pat. No. 8,007,768 B1) describe a core-shell pharmaceutical composition of nanoparticles for oral delivery. The shell consists of positively charged chitosan, and the core is a composition chitosan, transition metal ion, a negatively charged substrate, and at least a bioactive agent. The nanoparticles are formed via ionic gelation process, where the pH of PGA is 7.4 and pH of chitosan is 6.0. In some cases PGA complexone conjugate was used for the formation of nanoparticles and it was chelated to gadolinium. The complexone is DTPA, which is covalently bound to the biopolymer trough a linker.

Sung et al. (U.S. Pat. No. 8,048,404 B1, U.S. Pat. No. 8,048,453 B1) describe a pharmaceutical composition of bioactive nanoparticles composed of chitosan and poly-glutamic acid and a bioactive agent for oral delivery. The nanoparticles are characterizes with a positive surface charge, due to the positively charged chitosan dominately held on the surface of particles. The nanoparticles are formed via ionic gelation process, where the pH of PGA is 7.4 and pH of chitosan is 6.0. In some cases PGA complexone conjugate was used for the formation of nanoparticles and it was chelated to gadolinium. The complexone is DTPA, which is covalently bound to the biopolymer trough a linker

Borbely et al. (U.S. Pat. No. 7,976,825 B2) describe targeted macromolecular MRI contrast agent. The nanoparticles are formed by self-assembly of chitosan and poly-gamma-glutamic acid making a complex with paramagnetic ions. Folic acid is conjugated to the nanoparticles, therefore they are suitable for targeted delivery. The nanoparticles are formed via ionic gelation process, where the pH of polymer solutions is 3.0. There is no complexing agent in the invention, and the nanoparticle formulation is carried out at low (3.0) pH.

Li et al. (US 2007/0196275) describe conjugate molecules comprising a C225 ligand covalently bounded to a polymer, a metal chelating agent bonded to the polymer and a radioisotope chelated to the chelating agent. The polymer could be chitosan, or poly-glutamic acid, and chelatig agent is selected from DTPA, DOTA, EDTA, etc.

Sung et al. (U.S. Pat. No. 7,291,598 B2) describe nanoparticles of chitosan, PGA and at least 1 bioactive agent for paracellular drug delivery with a positive surface charge. The nanoparticles are formed with ionic gelation method. Nanoparticles are spherical in shape, and the chitosan dominates on the surface, able to open the tight junctions between Caco-2 cells. The patent also provides nanoparticles, where chitosan is cross-linked In this patent, there is no targeting and no paramagnetic ion transport via these nanoparticles.

Prokop et al. (WO 2004/096998) describe biocompatible, nanoparticulate formulations that are designed to retain and deliver peptides. Nanoparticles are obtained in a core-shell structure, where the core comprises at least 1 polyanionic polymer and a drug or therapeutic peptide, which is conjugated or crosslinked to a polymer and where the corona consists at least 1 polycationic polymer and a targeting ligand, which is cross-linked to or conjugated to a polymer. The polyanion could be poly(glutamic acid), and the polycation could be chitosan. The nanoparticle may comprise an inorganic salt and/or a bioluminescence agent or a contrast agent (macromolecular contr. agent) in the core and/or cation in the corona. One of the main point of this invention is that both of core and shell parts of the nanoparticles contain a polymer, which is cross-linked or conjugated to drug or therapeutic peptide in the core or targeting moiety in the shell.

### SUMMARY OF THE INVENTION

The present invention is directed to a novel targeting contrast agent for magnetic resonance imaging.

In some embodiments, the present invention provides targeting MR contrast agent diagnostic composition comprising (i) at least two polyelectrolyte biopolymers, (ii) a targeting agent conjugated to a polyelectrolyte biopolymer, (iii) a paramagnetic ligand complexed to the polyelectrolyte biopolymer, and optionally (iv) a complexing agent attached to the polyelectrolyte biopolymer.

More particularly, the self-assembled nanoparticles comprise at least two polyelectrolyte biopolymers, where at least one of the polyelectrolyte biopolymers is a polycation and the other of them is a polyanion biopolymer. The nanoparticles have been constructed by self-assembly of polyanion and polycation biopolymers based on the ion-ion interactions between their functional groups in aqueous media. The targeting moieties are conjugated to one of the self-assembled polyelectrolytes to realize a targeted delivery of particles as contrast agent. The paramagnetic ligands are complexed to one of the polyelectrolytes, via the carboxyl groups of polyanion or complexone ligands conjugated to the polycation biopolymer.

Polyelectrolyte biomacromolecules and their derivatives may form stable particles, deliver paramagnetic ions, thus increasing the molecular relaxivity of carriers. The present invention also relates to the composition and method for formation of biopolymer-based nanodevices for targeted delivery of MRI contrast agent.

Also provided are methods for making the contrast agent compositions that includes several steps described below: (i) the step of conjugating of targeting ligands to one of the polyelectrolyte biopolymers, (ii) the step of attaching the complexone ligand to the polycation biopolymer, (iii) the step of the self-assembly of polyelectrolyte biopolymers to form stable, targeting nanocarriers, and (iv) making a complex between the nanoparticles and paramagnetic ligand. The order of these steps of the nanoparticle formation can be modulated.

Formation of self-assembled contrast agent composition may be influenced by several conditions, such as the pH and the concentration of the solutions, the ratio of polyelectrolytes, the order of mixing, and the ratio of paramagnetic ligands.

In a preferred embodiment, one of the polyelectrolyte biopolymers is polycation, which is preferably chitosan; and the other of the polyelectrolyte biopolymers is polyanion, which is preferably poly-gamma-glutamic acid.

In a further embodiment, the chitosan of the nanoparticles ranges in molecular weight from about 20 kDa to 600 kDa, and the poly-gamma-glutamic acid of the nanoparticles ranges in molecular weight from about 50 kDa to 2500 kDa.

In a preferred embodiment, the degree of deacetylation of chitosan ranges between 40% and 99%.

In a preferred embodiment, the targeting agent is preferably folic acid, LHRH, RGD.

Preferable complexing agents include, but are not limited to: diethylenetriaminepentaacetic acid (DTPA), 1,4,7,10-tetracyclododecane-N,-N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethyl ether)N,N,N′,N′,-tetraacetic acid (EGTA), 1,4,8,11-tetraazacyclotradecane-N,N′,N″,N′″-tetraacetic acid (TETA), 1,4,7-triazacyclononane-N,N′,N″-triacetic acid (NOTA), or their reactive derivatives.

In a preferred embodiment, the paramagnetic ligand is preferably lanthanide or transition metal ion.

In a further embodiment, the nanoparticles have a mean particle size between about 30 and 500 nm, preferably between about 50 and 400 nm, and most preferably between 70 and 250 nm.

The present invention is directed to MRI contrast agent or diagnostic composition thereof comprising self-assembled polyelectrolyte biopolymers, targeting agent, paramagnetic ligand and optionally complexing agent. These self-assembled particles internalize into the targeted tumor cells due to the presence of targeting ligands. The internalized paramagnetic contrast agent enhances relaxivity, improve the signal-to-noise and therefore facilitate the early tumor diagnosis.

### BRIEF DESCRIPTION OF THE DRAWINGS

FIG. 1a shows the schematic representation of the formation process of targeting, paramagnetic contrast agent. Folated polyanion and polycation-complexone was mixed, and after that Gd-ion was added to produce targeting, paramagnetic nanoparticles, as MRI contrast agent.

FIG. 1b shows the schematic representation of the formation process of targeting, paramagnetic contrast agent. Folated polyanion and polycation was mixed, and after that Gd-ion was added to produce targeting, paramagnetic nanoparticles, as MRI contrast agent.

FIG. 1c shows the schematic representation of the formation process of targeting, paramagnetic contrast agent. Polyanion and folated polycation-complexone was mixed, and after that Gd-ion was added to produce targeting, paramagnetic nanoparticles, as MRI contrast agent.

FIG. 2 shows the size distribution of nanoparticulate contrast agent by volume in which nanocarriers were constructed by self-assembly of biopolymers at a concentration of 0.3 mg/ml, at given ratios, where the CH-DTPA solution was added into the PGA-FA solution, and after that the complex with gadolinium ions (c=0.4 mg/ml) was made.

FIG. 3 shows the T₁-weighted MRI images of two different targeted nanoparticular contrast agent (a,b) and distilled water (c). The targeted nanoparticular contrast agents was formed by mixing of PGA-FA and CH-DTPA and after that the complex with Gd-ions was performed. The difference between the two targeted contrast agent is the molecular weight of chitosan.

FIG. 4 shows the MTT assay results of novel, (2PGA-FA:1CH-EDTA+0.4Gd) targeted paramagnetic contrast agents measured on HeLa A2780 Jimt-1 KB MCF-7 HaCat cell lines.

FIG. 5 shows flow cytometric analysis of HeLa cells (a) and fluorescence intensity (FI) of HeLa cells (b) treated with novel nanoparticulate targeting MRI contrast agents (2PGA-FA:1CH-DTPA+0.4Gd) via flow citometric analysis.

FIG. 6 shows the in vitro cell proliferation. HeLa cells were cultured in the presence of 2PGA-FA:1CH-Gd targeted nanoparticular contrast agent.

FIG. 7 shows confocal microscopic images of HeLa cell treated with nanoparticles in which nanoparticles were constructed by self-assembly of folated poly-gamma-glutamic acid and chitosan-DTPAconjugate biopolymers at a ratio of 2:1 and at a concentration of 0.3 mg/ml, where the CH-DTPA solution was added into the PGA-FA solution, and after that the complex with gadolinium ions (c=0.4 mg/ml) was made. (Notation: 2PGA-FA:1CH-DTPA+0.4Gd).

FIG. 8 shows confocal microscopic images of JIMT-1 cell treated with nanoparticles in which nanoparticles were constructed by self-assembly of folated poly-gamma-glutamic acid and chitosan-DTPAconjugate biopolymers at a ratio of 2:1 and at a concentration of 0.3 mg/ml, where the CH-DTPA solution was added into the PGA-FA solution, and after that the complex with gadolinium ions (c=0.4 mg/ml) was made. (Notation: 2PGA-FA:1CH-DTPA+0.4Gd).

FIG. 9 shows confocal microscopic images of A2780 cell treated with nanoparticles in which nanoparticles were constructed by self-assembly of folated poly-gamma-glutamic acid and chitosan-DTPAconjugate biopolymers at a ratio of 2:1 and at a concentration of 0.3 mg/ml, where the CH-DTPA solution was added into the PGA-FA solution, and after that the complex with gadolinium ions (c=0.4 mg/ml) was made. (Notation: 2PGA-FA:1CH-DTPA+0.4Gd).

FIG. 10 shows the T₁-weighted MR images of the control HeLa cells (a), HeLa cell suspensions incubated with non-targeted 2PGA:1CH-Gd (b) and with folate-targeted 2PGA-FA:1CH-Gd nanoparticles (c).

FIG. 11 shows the T₁-weighted MR images of the control Jimt-1 cells (a), and Jimt-1 cell suspensions incubated with folate-targeted 2PGA-FA:1CH-DOTA+0.4Gd nanoparticles (b).

FIG. 12. MRI study on the uptake of 2PGA-FA:1CH-Gd contrast agent into HeLa cancer xenografts. T1 weighted MR images of CD1 female nude mice bearing subcutaneous HeLa. The increase in signal intensity of 2PGA-FA:1CH-Gd contrast agent can be visualized by an increase of red color in the color pixel map in treated mice. Much less effect is observed in the control tumor.

FIG. 13. MRI study on the uptake of 2PGA-FA:1CH-DTPA+0.4Gd contrast agent into HeLa cancer xenografts. In vivo T1 MR image of tumor bearing control animal (a), and animal treated with 2PGA-FA:1CH-DTPA+0.4Gd contrast agent.

FIG. 14 MRI study on the uptake of 2PGA-FA:1CH-DOTA+0.4Gd contrast agent into HeLa cancer xenografts. In vivo T1 MR image of tumor bearing control animal (a), and animal treated with 2PGA-FA:1CH-DOTA+0.4Gd contrast agent.

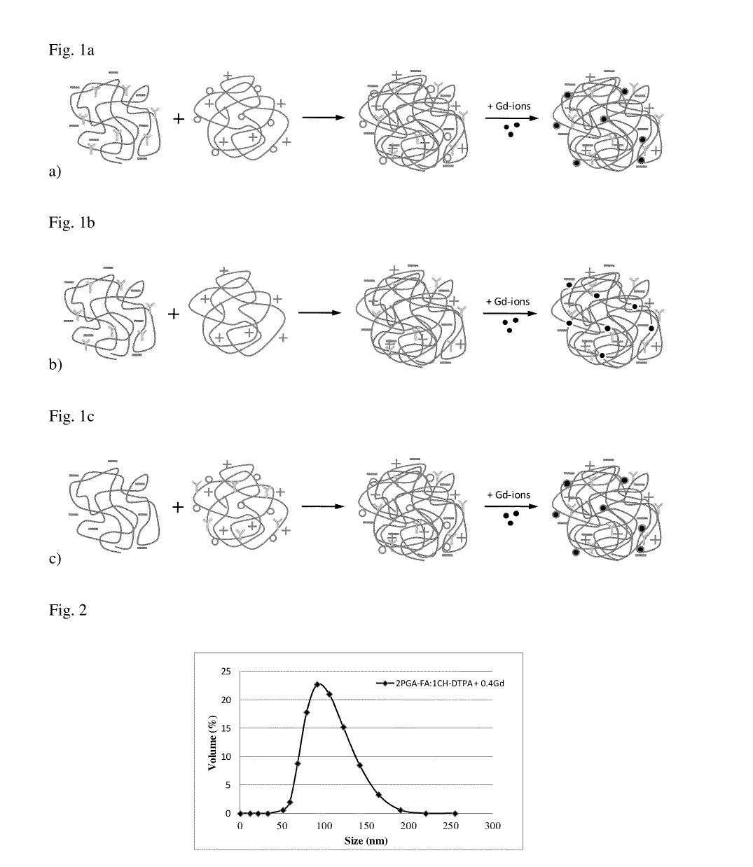

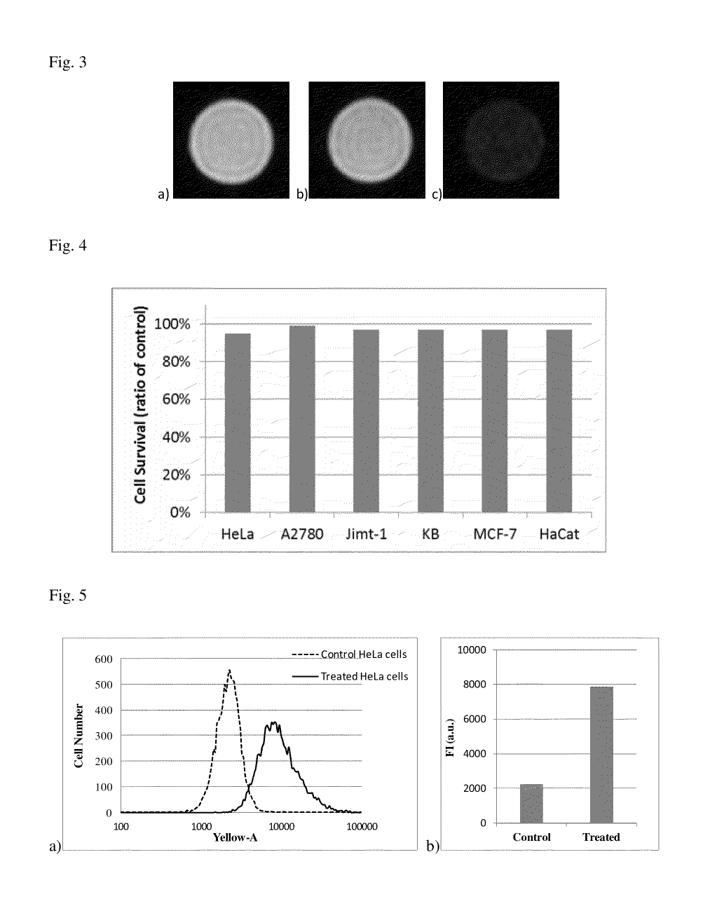

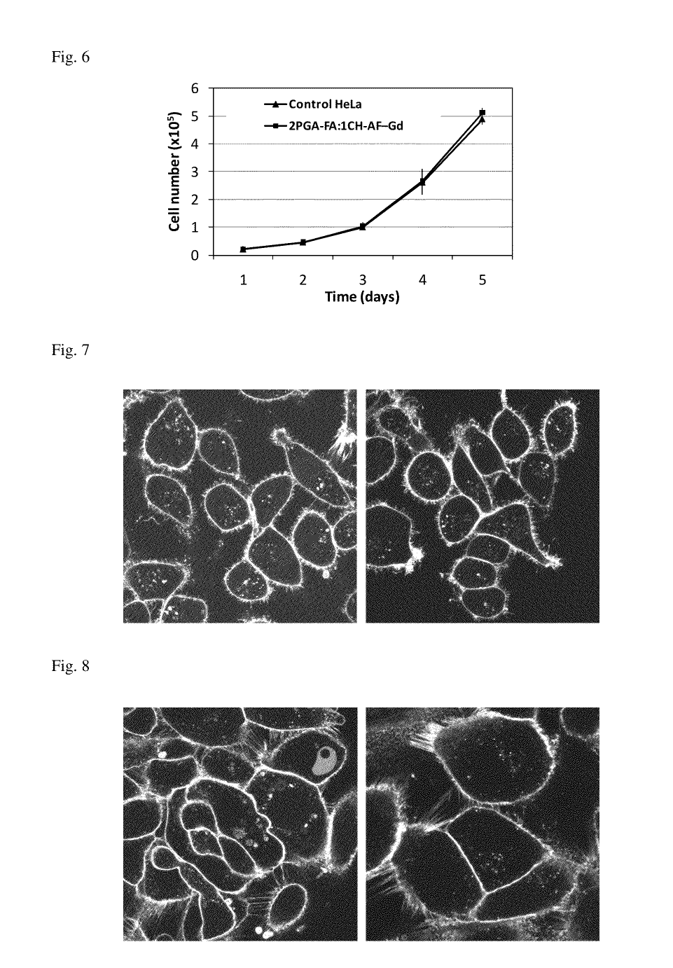

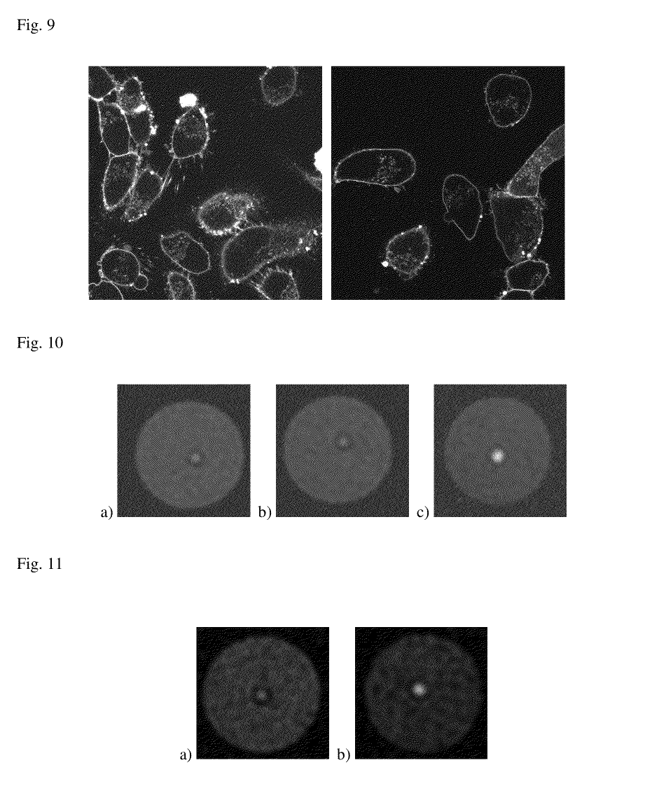

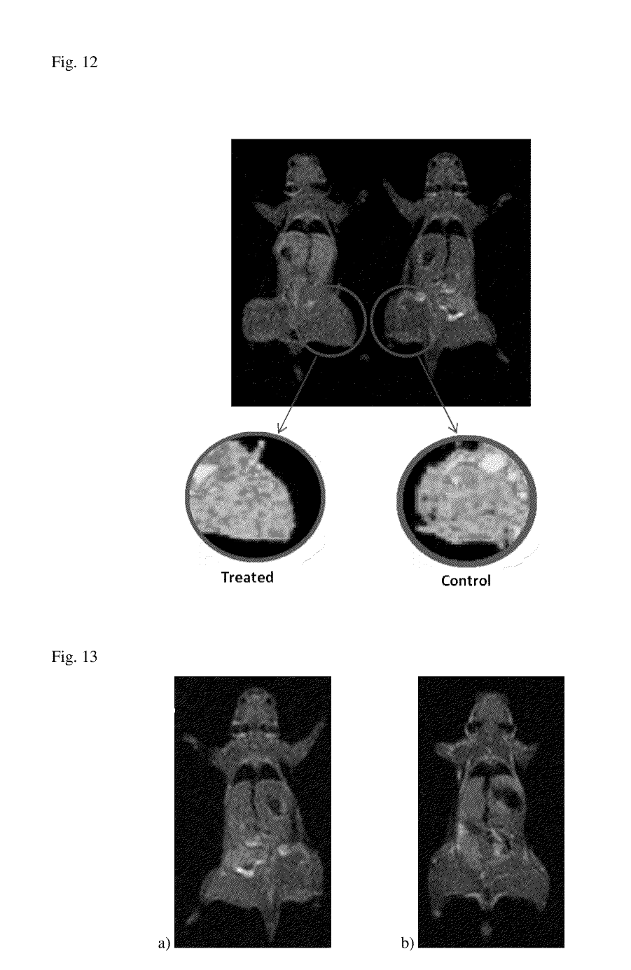

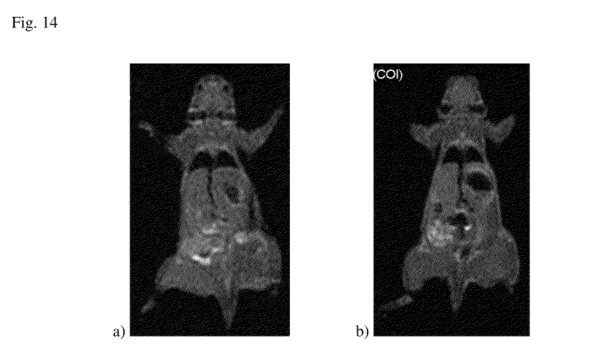

### DETAILED DESCRIPTION OF THE INVENTION

The present invention provides novel, targeting, paramagnetic contrast agent for magnetic resonance imaging (MRI) and method for forming them for targeted delivery of paramagnetic ligands. In preferred embodiments, self-assembled particles are provided as contrast agent for MRI, labeled with targeting moieties, paramagnetic ligands, and optionally complexone ligands conjugated to a biopolymer. These particles enhance relaxivity, improve the signal-to-noise and are able for targeted delivery. Methods for making these targeting MRI contrast agent are also provided.

### Nanoparticles, as Contrast Agent Compositions

The present invention is directed to biocompatible, biodegradable, polymeric nanoparticles, as paramagnetic contrast agent, formed by self-assembly via ion-ion interaction of oppositely charged functional groups of polyelectrolyte biopolymers. The nanoparticles of the present invention contain paramagnetic metal ions.

In a preferred embodiment, the paramagnetic ions are preferably lanthanide or transition metal ions, more preferably gadolinium-, manganese-, chromium-ions, most preferably gadolinium ions, useful as MRI contrast agent.

In a preferred embodiment, the paramagnetic ions are homogeneously distributed throughout the self-assembled nanoparticle. The paramagnetic ions can make stable complexes with the carboxyl functional groups of polyanion, which is self-assembling into the nanoparticles. In a further embodiment, the paramagnetic ions can make stable complex with the complexone ligands attached to the polycation biopolymer, therefore they could be performed homogeneously dispersed.

In a preferred embodiment, the biopolymers are water-soluble, biocompatible, biodegradable polyelectrolyte biopolymers. One of the polyelectrolyte biopolymers is a polycation, positively charged polymers, which is preferably chitosan or its derivatives. The other of the polyelectrolyte biopolymers is a polyanion, a negatively charged biopolymer. The polyanion is preferably selected from a group consisting of polyacrylic acid (PAA), poly-gamma-glutamic acid (PGA), hyaluronic acid (HA), and alginic acid (ALG).

In a preferred embodiment, the polycation of the nanoparticles ranges in molecular weight from about 20 kDa to 600 kDa, and the polyanion of the nanoparticles ranges in molecular weight from about 50 kDa to 2500 kDa.

In a preferred embodiment, the degree of deacetylation of chitosan ranges between 40% and 99%.

The nanoparticle compositions described herein, contain cationic biopolymer or its derivatives meaning complexone conjugate, anionic biopolymer, targeting agent, and paramagnetic ion.

In a preferred embodiment, the targeting agent is coupled covalently to one of the biopolymers using carbodiimide technique in aqueous media. Water soluble carbodiimide, as coupling agent forms amide bonds between the carboxyl and amino functional groups, therefore the targeting ligand could be covalently bound to one of the polyelectrolyte biopolymers.

In the present invention, the preferred targeting agent is selected from folic acid, LHRH, RGD.

In a preferred embodiment, the most preferred targeting agent is folic acid, which facilitates the folate mediated uptake of nanoparticles, as tumor specific contrast agents. The nanoparticles of the present invention are preferably targeted to tumor and cancer cells, which overexpress folate receptors on their surface. Due to the binding activity of folic acid ligands, the nanoparticles selectively link to the folate receptors held on the surface of targeted tumor cells, internalize and accumulate in the tumor cells.

In a preferred embodiment, self-assembled nanoparticles comprising of polyanion biopolymer, polycation biopolymer, targeting agent covalently attached to one of the biopolymers and paramagnetic ions complexed to the functional carboxyl groups of polyanion biopolymer are provided. In a further embodiment, self-assembled nanoparticles comprising of polyanion biopolymer, polycation biopolymer, complexone ligand covalently coupled to the polycation, targeting agent covalently attached to one of the biopolymers and paramagnetic ions complexed to the self-assembled nanoparticles via the complexone ligand are provided.

In a preferred embodiment, the complexing agent is coupled covalently to the polycation biopolymer. Water-soluble carbodiimide, as coupling agent is used to make stable amide bonds between the carboxyl and amino functional groups in aqueous media. Using reactive derivatives of complexing agents (e.g. succinimide, thiocyanete), the polycation-complexone conjugate can be directly formed in one-step process without any coupling agents. The nanoparticles can make stable complex with the paramagnetic ions through these complexone ligans.

In a preferred embodiment, the complexing agents are preferably diethylenetriaminepentaacetic acid (DTPA), 1,4,7,10-tetracyclododecane-N,-N′,N″,N″′-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethyl ether)N,N,N′,N′,-tetraacetic acid (EGTA), 1,4,8,11-tetraazacyclotradecane-N,N′,N″,N′″-tetraacetic acid (TETA), 1,4,7-triazacyclononane-N,N′,N″-triacetic acid (NOTA) or their reactive derivatives. More preferably, the complexing agents are DOTA, DTPA, EDTA and NOTA, most preferably DTPA.

In a preferred embodiment, the nanoparticles described herein have a hydrodynamic diameter between about 30 and 500 nm, preferably between about 50 and 400 nm, and the most preferred range of the hydrodynamic size of nanoparticles is between 70 and 250 nm.

### Methods of Making Nanoparticles, as Contrast Agent Compositions

The present invention is directed to novel, biocompatible, biodegradable, targeting nanoparticles as MRI contrast agent. The nanoparticle compositions described herein are prepared by self-assembly of oppositely charged polyelectrolytes via ion-ion interaction between their functional groups. The targeting ligands are conjugated covalently to one of the polyelectrolyte biopolymers and paramagnetic ion is complexed thereto, as MRI active ligands. The paramagnetic ions can make complexes with the nanoparticles via the carboxyl groups of polyanions or optionally via complexing agents covalently conjugated to the polycation biopolymer.

In a preferred embodiment, the targeting ligand is attached to one of the biopolymers covalently. The targeting agent is preferably folic acid, LHRH, RGD, the most preferably folic acid.

The polyanions via their reactive carboxyl functional groups can form stable amide bond with the amino functional groups of folic acid using carbodiimide technique. The plycations via their reactive amino functional groups can form stable amide bond with the carboxyl functional groups of folic acid. In the present invention, folated biopolymers meaning folated polyanion or folated polycation can be used for the formation of nanoparticles, as targeted paramagnetic MRI contrast agent.

In a preferred embodiment, the polycation or its derivatives are used for the formation of nanoparticles. In the preferred embodiment derivatives of polycation are produced by coupling complexing agent to it covalently. Water soluble carbodiimide is used as coupling agent to form stable amide linkage between the amino groups of polycation and carboxyl groups of complexing agent. Using reactive derivatives of complexing agents (e.g. succinimide, thiocyanete), the polycation-complexone conjugate can be directly formed in one-step process without any coupling agents. In the present invention several complexing agent having reactive carboxyl groups are used to make stable complex with paramagnetic ions and therefore afford possibility to use these systems as MRI contrast agent.

In a preferred embodiment, four types of polycations can be used for the formation of nanoparticles: (i) polycation without any covalent modification; (ii) targeted polycation, when the targeting agent is coupled covalently to the polycation; (iii) polycation-complexone conjugate, when the complexing agent is covalently attached to the polycation; and (iv) targeted polycation-complexone conjugate, when targeting moiety and the complexing agent are covaletly coupled to the polycation biopolymer.

In case of the carbodiimide technique, preparation of polycation-complexone conjugate can be obtained in different order of steps. In a preferred embodiment, a polycation can be solubilized in an aqueous solution of complexing agent, and then covalent linkages are performed by addition of water soluble carbodiimide solution. In a further embodiment, the aqueous solution of complexing agent was added to the polycation solution, and then the covalent amide bonds were performed by addition of water soluble carbodiimide dropwise.

Using reactive derivatives of the complexing agents (e.g. succinimide, thiocyanete), the polycation-complexone conjugate can be directly formed in one-step process without any coupling agents.

In a preferred embodiment, for the formation of conjugation, the concentration of biopolymer ranges between about 0.05 mg/ml and 5 mg/ml, preferably 0.1 mg/ml and 2 mg/ml, and the most preferably 0.3 mg/ml and 1 mg/ml.

In a preferred embodiment, preparation of targeted polycation complexone-conjugate can be obtained in different order of steps. In a preferred embodiment, targeting ligand is coupled to the polycation, and after that complexing agent is reacted with the targeted polycation to produce targeted polycation conjugate composition. In further embodiment, the complexing agent is coupled to the polycation, and after that targeting ligand is attached to the polycation-complone conjugate to produce the targeted polycation conjugate composition.

In a preferred embodiment, the overall degree of substitution of complexing agent in polycation-complexone conjugate is generally in the range of about 1-50%, preferably in the range of about 5-30%, and most preferably in the range of about 10-20%.

The nanocarrier formation of the present invention can be obtained in different order of steps.

In a preferred embodiment, nanoparticles can be produced from the reaction, whereby solutions of targeted polyanion and solution of polycation or polycation-complexone are mixed to form stable, self-assembled nanoparticles, and after that aqueous solution of paramagnetic ions is added to these nanoparticles to make stable nanoparticulate complex. In a further embodiment, aqueous solution of polyanion and targeted polycation or targeted polycation-complexone conjugate are mixed to form stable, self-assembled nanoparticles and after that aqueous solution of paramagnetic ions is added to these nanoparticles to make stable nanoparticulate complex.

The nanoparticles can be formed independently of order of addition. In a preferred embodiment a polycation or its derivatives and a polyanion or its derivatives are mixed to produce stable nanoparticles, and after that making complex with paramagnetic ions was performed. In a further embodiment the order of biopolymer mixing is modulated. In a further embodiment, the first step is the making of complex, where paramagnetic ions are added to the biopolymer which can make stable complex therewith, and after that this complex composition is mixed with the oppositely charged biopolymer for the nanoparticle formation. In consideration of nanoparticles formation, order of addition of polyelectrolytes is not a main factor.

The nanoparticle compositions described herein are prepared by mixing aqueous solutions of the polyanion or modified polyanion, the polycation or modified polycation and the paramagnetic ion at given ratios and orders of addition. In a preferred embodiment, the concentration of biopolymers ranges between about 0.005 mg/ml and 2 mg/ml, preferably between 0.2 mg/ml and 1 mg/ml, most preferably 0.3 mg/ml and 0.5 mg/ml. The concentration ratio of biopolymers mixed is about 2:1 to 1:2, most preferably about 1:1. The biopolymers are mixed in a weight ratio of 6:1 to 1:6, most preferably 3:1 to 1:3.

The size of nanoparticles can be controlled by several reaction conditions, such as the concentration of biopolymers, the ratio of biopolymers, and the order of addition. The charge ratio of biopolymers depends on the pH of the environment. In preferred embodiment, the pH of polycation or its derivatives varied between 3.5 and 5.0, and the pH of aqueous solution of polyanion or its derivatives ranges between 7.5 and 9.5. The paramagnetic ion solution was used as simple aqueous solution without any pH adjusting.

At low pH, the polycation is in extended coil conformation due the repulsive interactions between the charged functional groups. At low pH, most of the functional groups of polycation are protonated, a polycation with high charge density can be performed.

At high pH, the polyanion is also in extended coil conformation. Most of its functional groups are in deprotonated form; therefore polyanion with high charge density can be obtained.

Stable nanoparticles are formed by self-assembly of biopolymers, as polyelectrolytes. The orientation of biopolymers in the nanoparticles could be statistical due to the high charge density of both types of macromolecules. Nevertheless the orientation of biopolymers in the nanoparticles can be influenced minimally by the order of addition.

In a preferred embodiment, biopolymers with high charge density form stable nanoparticles due to the given pH values. The surface charge of nanoparticles could be influenced by several reaction parameters, such as ratio of biopolymers, ratio of residual functional groups of biopolymers, pH of the biopolymers and the environment, etc. The electrophoretic mobility values of nanoparticles, showing their surface charge, could be positive or negative, preferably negative, depending on the reaction conditions described above.

In a preferred embodiment, nanoparticulate compositions, as targeted, paramagnetic MRI contrast agents are provided. The paramagnetic ligand is preferably lanthanide or transition metal ions, more preferably gadolinium-, manganese-, chromium-ions, most preferably gadolinium ions, useful for MRI. The preferred paramagnetic ions can make stable complex with the targeting, self-assembled nanoparticles due the residual carboxyl functional groups of polyanion or due to the complexing agents, which are covalently conjugated to polycation.

In a preferred embodiment, gadolinium-chloride solution was used as simple aqueous solution without any pH adjusting. In a preferred embodiment, concentration of gadolinium ion ranges between about 0.2 mg/ml and 1 mg/ml, most preferably between 0.4 mg/ml and 0.5 mg/ml. The molar ratio of said gadolinium ions and complexone conjugated to the polycation ranges preferably between 1:10 and 1:1, more preferably 1:5 and 1:1, and most preferably 1:1.

### Methods of Using Nanocarrier Compositions

The nanoparticle composition is useful for targeted delivery of paramagnetic ligand. The present invention is directed to methods of using the above-described nanoparticles, as targeted, paramagnetic MRI contrast agent.

In a preferred embodiment, the nanoparticles as nanocarriers deliver the paramagnetic ligands to the targeted tumor cells in vitro, therefore can be used as targeted, paramagnetic MRI contrast agents. The nanoparticulate MRI contrast agent internalizes and accumulates in the targeted tumor cells, which overexpress folate receptors, to facilitate the early tumor diagnosis. The side effect of these contrast agents is minimal, because of the receptor mediated uptake of nanoparticles.

In a preferred embodiment, the paramagnetic contrast agents are stable at pH 7.4, it may be injected intravenously. Based on the blood circulation, the nanoparticles could be transported to the area of interest.

The ability of the particles to be internalized was studied in cultured cancer cells, which overexpresses folate receptors using confocal microscopy and flow cytometry. Due to the folic acid, as targeting moiety, the nanoparticles efficiently internalize into the targeted tumor cells, which overexpress folate receptors. The use of targeted, paramagnetic nanoparticles, as MRI contrast agent enhances the receptor mediated uptake, therefore these nanoparticles can be attractive candidates as contrast agents for magnetic resonance imaging.

### EXAMPLES

### Example 1

### Preparation of Folated Poly-Gamma-Glutamic Acid (γ-PGA)

Folic acid was conjugated via the amino groups to γ-PGA using carbodiimide technique. γ-PGA (m=60 mg) was dissolved in water (V=100 ml) to produce aqueous solution. The pH of the polymer solution was adjusted to 6.0. After the dropwise addition of cold water-soluble 1-[3-(dimethylamino)propyl]-3-ethylcarbodiimide methiodide (CDI) (m=13 mg in 2 ml distilled water) to the γ-PGA aqueous solution, the reaction mixture was stirred at 4° C. for 1 h, then at room temperature for 1 h. After that, folic acid (m=22 mg in dimethyl sulfoxide, V=10 ml) was added droppwise to the reaction mixture and stirred 4° C. for 1 h, then at room temperature for 24 h. The folated poly-γ-glutamic acid (γ-PGA-FA) was purified by dialysis.

### Example 2

### Preparation of Folated Chitosan

A solution of 1-(3-dimethylaminopropyl)-3-ethylcarbodiimide hydrochloride (CDI) and FA in anhydrous DMSO was prepared and stirred at room temperature until FA was well dissolved (1 h). Chitosan was dissolved in 0.1M hydrochloric acid, to produce a solution with a concentration of 1 mg/ml, and then adjusted to pH 5.5 with 0.10 M sodium hydroxide solution. After the dropwise addition of CDI (m=5.1 mg in 1 ml distilled water) to the chitosan solution (V=20 ml), the reaction mixture was stirred for 10 min Then folic acid (m=8.5 mg in dimethyl sulfoxide, V=1 ml) was added to the reaction mixture. The resulting mixture was stirred at room temperature in the dark for 24 h. It was brought to pH 9.0 by drop wise addition of diluted aqueous NaOH and was washed three times with aqueous NaOH, and once with distilled water. The polymer was isolated by lyophilization.

### Example 3

### Preparation of Chitosan-DTPA Conjugate

Chitosan (m=15 mg) was solubilized in water (V=15 ml); its dissolution was facilitated by dropwise addition of 0.1M HCl solution. After the dissolution, the pH of chitosan solution was adjusted to 5.0. After the dropwise addition of DTPA aqueous solution (m=11 mg, V=2 ml, pH=3.2), the reaction mixture was stirred at room temperature for 30 min, and at 4° C. for 15 min after that, CDI (m=8 mg, V=2 ml distilled water) was added dropwise to the reaction mixture and stirred at 4° C. for 4 h, then at room temperature for 20 h. The chitosan-DTPA conjugate (CH-DTPA) was purified by dialysis.

### Example 4

### Preparation of Chitosan-EDTA Conjugate

Chitosan (m=15 mg) was solubilized in water (V=15 ml); its dissolution was facilitated by dropwise addition of 0.1M HCl solution. After the dissolution, the pH of chitosan solution was adjusted to 5.0. After the dropwise addition of EDTA aqueous solution (m=11 mg, V=2 ml, pH=6), the reaction mixture was stirred at room temperature for 30 min, and at 4° C. for 15 min after that, CDI (m=8 mg, V=1 ml distilled water) was added dropwise to the reaction mixture and stirred at 4° C. for 4 h, then at room temperature for 20 h. The chitosan-EDTA conjugate (CH-EDTA) was purified by dialysis.

### Example 5

### Preparation of Chitosan-DOTA Conjugate

Chitosan (m=15 mg) was solubilized in water (V=15 ml); its dissolution was facilitated by dropwise addition of 0.1M HCl solution. After the dissolution, the pH of chitosan solution was adjusted to 5.0. After the dropwise addition of DOTA aqueous solution (m=11 mg, V=2 ml), the reaction mixture was stirred at room temperature for 30 min, and at 4° C. for 15 min. after that, CDI (m=8 mg, V=2 ml distilled water) was added droppwise to the reaction mixture and stirred 4° C. for 4 h, then at room temperature for 20 h. The chitosan-DOTA conjugate (CH-DOTA) was purified by dialysis.

### Example 6

### Preparation of Self-Assembled Nanoparticulate Contrast Agent

Stable self-assembled nanoparticles were developed via an ionotropic gelation process between the folated poly-γ-glutamic acid (γ-PGA-FA), and chitosan-DTPA conjugate (CH-DTPA) and Gd-ions. Briefly, CH-DTPA solution (c=0.3 mg/ml, V=1 ml, pH=4.0) was added into γ-PGA-FA solution (c=0.3 mg/ml, V=2 ml, pH=9.5) under continuous stirring. An opaque aqueous colloidal system was gained, which remained stable at room temperature for several weeks at physiological pH. To make complex with Gd³⁺, a solution of Gd(III)-chloride (c=0.4 mg/ml, V=400 μl) was added dropwise to the aqueous colloid system containing targeted self-assembled nanoparticles (γ-PGA-FA/CH-DTPA-Gd) and stirred at room temperature for 30 min

### Example 7

### Preparation of Self-Assembled Nanoparticulate Contrast Agent

Stable self-assembled nanoparticles were developed via an ionotropic gelation process between the folated poly-γ-glutamic acid (γ-PGA-FA), and chitosan-DOTA conjugate. Briefly, CH-DOTA solution (c=0.3 mg/ml, V=1 ml, pH=4.0) was added into γ-PGA-FA solution (c=0.3 mg/ml, V=2 ml, pH=9.5) under continuous stirring. An opaque aqueous colloidal system was gained (pH 7.4), which remained stable at room temperature for several weeks at physiological pH. To make complex with Gd³⁺, a solution of Gd(III)-chloride (c=0.4 mg/ml, V=400 μl) was added dropwise to the aqueous colloid system containing targeted self-assembled nanoparticles (γ-PGA-FA/CH-DOTA-Gd) and stirred at room temperature for 30 min.

### Example 8

### Preparation of Self-Assembled Nanoparticulate Contrast Agent

Stable self-assembled nanoparticles were developed via an ionotropic gelation process between the folated poly-γ-glutamic acid (γ-PGA-FA), and chitosan-EDTA and Gd-ions. Briefly, CH-EDTA solution (c=0.1 mg/ml, V=1 ml, pH=4.0) was added into γ-PGA-FA solution (c=0.1 mg/ml, V=3 ml, pH=9.5) under continuous stirring. An opaque aqueous colloidal system was gained, which remained stable at room temperature for several weeks at physiological pH. To make complex with Gd³⁺, a solution of Gd(III)-chloride (c=0.4 mg/ml, V=400 μl) was added dropwise to the aqueous colloid system containing targeted self-assembled nanoparticles (γ-PGA-FA/CH-Gd) and stirred at room temperature for 30 min.

### Example 9

### Preparation of Self-Assembled Nanoparticulate Contrast Agent

Stable self-assembled nanoparticles were developed via an ionotropic gelation process between the folated poly-γ-glutamic acid (γ-PGA-FA), chitosan and Gd-ions. Briefly, CH solution (c=0.3 mg/ml, V=1 ml, pH=5.0) was added into γ-PGA-FA solution (c=0.3 mg/ml, V=3 ml, pH=8.0) under continuous stirring. An opaque aqueous colloidal system was gained, which remained stable at room temperature for several weeks at physiological pH. To make complex with Gd³⁺, a solution of Gd(III)-chloride (c=0.4 mg/ml, V=400 μl) was added dropwise to the aqueous colloid system containing targeted self-assembled nanoparticles (γ-PGA-FA/CH-Gd) and stirred at room temperature for 30 min

### Example 10

### Characterization of Self-Assembled Nanoparticulate Contrast Agent

The transmittances of solutions containing self-assembled paramagnetic nanoparticulate complexes were measured using Hitachi U-1900 ultraviolet spectrophotometer at an operating wavelength of λ=500 nm in optically homogeneous quartz cuvettes. The morphological characterization of self-assembled nanoparticle-gadolinium conjugate nanoparticles was carried out with a JEOL2000 FX-II transmission electron microscope. The sample was prepared by placing a drop of the nanoparticle solution onto a 400 mesh copper grid coated with carbon. The hydrodynamic size and size distribution of particles was measured using a dynamic light scattering (DLS) technique with a Zetasizer Nano ZS (Malvern Instruments Ltd., Grovewood, Worcestershire, UK). This system is equipped with a 4 mW helium/neon laser with a wavelength of 633 nm and measures the particle size with the noninvasive backscattering technology at a detection angle of 173°. Particle size measurements were performed using a particle-sizing cell in the automatic mode. The mean hydrodynamic diameter was calculated from the autocorrelation function of the intensity of light scattered from the particles. Electrokinetic mobility of the nanoparticles was measured in folded capillary cell (Malvern) with a Zetasizer Nano ZS apparatus.

### Example 11

### Magnetic Resonance Imaging

Signal intensity of the nanoparticulate contrast agents was measured using a clinical 1.5 T Signa LX MR scanner at room temperature. For the measurement, the T₁-weighted scans were performed with 420.0 ms of repetition time (TR) and 20.0 ms of echo time (TE); thickness was 1.5 mm and space was 0. T₁ relaxation time values were calculated from signal intensities. For the measurements, inversion time values were 50, 100, 200, 400, 800, 1400, 2200 and 3600 ms, the TR=4000.0 ms and TE=9 ms; thickness: 2 mm and space: 1 mm were during the measurements.

### Example 12

### Cellular Uptake of Nanoparticulate Contrast Agent

Internalization and selectivity of nanoparticulate contrast agent was investigated in cultured human cancer cells overexpressing folate receptors by using confocal microscopy and flow cytometry. The samples were imaged on an Olympus FluoView 1000 confocal microscope. Excitation was performed by using the 488 nm line of an Ar ion laser (detection: 500-550 nm) and the 543 nm line of a HeNe laser (detection: 560-610 nm) to image Alexa 488 and Alexa 546 respectively. Images were analyzed using Olympus FV10-ASW 1.5 software package. Flow cytometric analysis (BD FACSArray Bioanalyzer System) was carried out with a single-cell suspension, and only the live cells were gated based on forward and side scatter dot plots.

The nanoparticles internalized and accumulated in the targeted tumor cells. Folic acid, as targeting agent is specific to cancer cells, which overexpress folate receptors. due to this targeting moiety, enhanced receptor mediated cellular uptake of the novel self-assembled nanoparticles can be observed. Therefore these nanoparticles can be attractive candidates as tumor specific contrast agents for magnetic resonance imaging.

1. A diagnostic nanocomposition applicable for magnetic resonance imaging (MRI) comprising (i) at least two, preferably water-soluble, biocompatible and biodegradable polyelectrolyte biopolymers, (ii) a targeting agent conjugated to a polyelectrolyte biopolymer, (iii) a paramagnetic ligand complexed to a polyelectrolyte biopolymer, and optionally (iv) a complexing agent attached to a polyelectrolyte biopolymer.

2. The diagnostic nanocomposition as claimed in claim 1, wherein at least one of the polyelectrolyte biopolymers is a polycation or a derivative thereof, preferably chitosan, and the other of them is a polyanion biopolymer or a derivative thereof, preferably selected from the group consisting of polyacrylic acid (PAA), poly-gamma-glutamic acid (PGA), hyaluronic acid (HA), and alginic acid (ALG), preferably poly-gamma-glutamic acid (PGA), said biopolymers being preferably self-assembled based on the ion-ion interactions between their functional groups.

3. The diagnostic nanocomposition as claimed in claim 1, wherein the paramagnetic ligands are complexed to one of the polyelectrolytes, via the carboxyl groups of the polyanion or its derivative conjugated to the polycation biopolymer.

4. The diagnostic nanocomposition as claimed in claim 1, wherein
a) the polycation, preferably the chitosan, has a molecular weight from about 20 kDa to 600 kDa, preferably the degree of deacetylation of chitosan ranges between 40% and 99%; said polycation optionally (i) being without any covalent modification; (ii) having the targeting agent coupled covalently to the polycation; (iii) being in the form of a polycation-complexone conjugate, when the complexing agent is covalently attached to the polycation; or (iv) being in the form of a polycation-complexone conjugate, where the targeting moiety and the complexing agent are covalently coupled to the polycation and/or
b) the polyanion, preferably the poly-gamma-glutamic acid (PGA) has a molecular weight from about 50 kDa to 2500 kDa; and/or
c) the targeting agent is selected from the group of folic acid, LHRH, RGD, preferably folic acid; and/or
d) the complexing agent is selected from the group consisting of diethylenetriaminepentaacetic acid (DTPA), 1,4,7,10-tetracyclododecane-N,-N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethyl ether)N,N,N′,N′,-tetraacetic acid (EGTA), 1,4,8,11-tetraazacyclotradecane-N,N′,N″,N′″-tetraacetic acid (TETA), 1,4,7-triazacyclononane-N,N′,N″-triacetic acid (NOTA) or their reactive derivatives, more preferably, the complexing agents are DOTA, DTPA, EDTA and NOTA, most preferably DTPA; and/or
e) the paramagnetic ligand is a lanthanide or a transition metal ion, preferably gadolinium-, manganese-, chromium-ion, most preferably gadolinium ion, optionally being complexed to the self-assembled nanoparticles via a complexone ligand, and preferably being homogeneously distributed throughout the self-assembled nanoparticle.

5. The diagnostic nanocomposition as claimed in claim 1, wherein the nanoparticles have a mean particle size between about 30 and 500 nm, preferably between about 50 and 400 nm, and most preferably between 70 and 250 nm.

6. The diagnostic nanocomposition as claimed in claim 1, wherein
a) the self-assembled nanoparticles are constructed by self-assembly of polyanion and polycation biopolymers based on the ion-ion interactions between their functional groups, preferably in an aqueous media; and/or
b) the targeting agent is covalently attached to one of the biopolymers, preferably by a coupling agent, preferably carbodiimide; and/or
c) the paramagnetic ion is complexed to the functional carboxyl groups of polyanion.

7. The diagnostic nanocomposition as claimed in claim 1, wherein the complexone ligand is covalently coupled to the polycation, the targeting agent is covalently attached to one of the biopolymers, and the paramagnetic ions are complexed to the self-assembled nanoparticles via the complexone ligand.

8. A process for the preparation of the diagnostic nanocomposition as claimed in claim 1, comprising the steps of (i) conjugating of the targeting ligands to one of the polyelectrolyte biopolymers, (ii) attaching the complexone ligand to the polycation biopolymer, (iii) the self-assembly of polyelectrolyte biopolymers to form stable, targeting nanocarriers, and (iv) making a complex between the nanoparticles and paramagnetic ligand, wherein steps (i) to (iv) can be made in any order.

9. The process as claimed in claim 8, wherein the concentration of the biopolymer used ranges between about 0.05 mg/ml and 5 mg/ml, preferably 0.1 mg/ml and 2 mg/ml, and most preferably 0.3 mg/ml and 1 mg/ml.

10. The process as claimed in claim 8, wherein the overall degree of substitution of complexing agent in polycation-complexone conjugate is in the range of about 1-50%, preferably in the range of about 5-30%, and most preferably in the range of about 10-20%.

11. The process as claimed in claim 8, wherein water soluble carbodiimide is used as coupling agent to form stable amide linkage between the amino groups of polycation and carboxyl groups of complexing agent.

12. The process as claimed in claim 8, wherein a reactive derivative, preferably the succinimide or thiocyanate of the complexing agent is used for the preparation of the polycation-complexone conjugate in one-step process without any coupling agents.

13. The process as claimed in claim 8, wherein the nanoparticles are produced from the reaction, whereby a solution, preferably aqueous solution of the targeted polyanion and a solution of the polycation or polycation-complexone are mixed to form nanoparticles, then an aqueous solution of paramagnetic ions is added to these nanoparticles to make stable nanoparticulate complex.

14. The process as claimed in claim 8, wherein the concentration of the biopolymers used ranges between about 0.005 mg/ml and 2 mg/ml, preferably between 0.2 mg/ml and 1 mg/ml, most preferably 0.3 mg/ml and 0.5 mg/ml.

15. The process as claimed in claim 8, wherein the concentration ratio of biopolymers mixed is about 2:1 to 1:2, most preferably about 1:1.

16. The process as claimed in claim 8, wherein the biopolymers are mixed in a weight ratio of 6:1 to 1:6, most preferably 3:1 to 1:3.

17. The process as claimed in claim 8, wherein the pH of the polycation used is between 3.5 and 5.0, and the pH of aqueous solution of polyanion used is between 7.5 and 9.5.

18. The process as claimed in claim 8, wherein a gadolinium-chloride solution is used as aqueous solution, wherein
a) the concentration of gadolinium ion ranges between about 0.2 mg/ml and 1 mg/ml, most preferably between 0.4 mg/ml and 0.5 mg/ml; and/or
b) the molar ratio of the gadolinium ions and complexone conjugated to the polycation ranges preferably between 1:10 and 1:1, more preferably 1:5 and 1:1, and most preferably 1:1.

19. A method for the targeted delivery of a paramagnetic ligand, said method comprising administering the nanocomposition according to claim 1 to a subject.

20. The method according to claim 19, wherein the compositions are injected intravenously.
